(19) United States
(12) Patent Application Publication
(10) Pub. No.: US 2020/0138748 A1
(43) Pub. Date: May 7, 2020

(54) COMPOSITION AND METHOD FOR AIDING SLEEP

(71) Applicant: Sequential Medicine Limited, Grand Cayman (KY)

(72) Inventor: Lan Bo CHEN, Lexington, MA (US)

(73) Assignee: Sequential Medicine Limited, Grand Cayman (KY)

(21) Appl. No.: 16/181,970

(22) Filed: Nov. 6, 2018

(51) Int. Cl. A61K 31/138; A61K 31/437; A61K 31/5513; A61K 9/50; A61K 9/20; A61P 25/20

(52) U.S. Cl. CPC A61K 31/138; A61K 31/437; A61P 25/20; A61K 9/5026; A61K 9/2081; A61K 31/5513

(57) ABSTRACT

Disclosed is a controlled-release formulation for treating disturbed sleep or insomnia in a subject. The formulation is formulated to release diphenhydramine HCl and a sleep aid in sequence at two stages.

### BACKGROUND OF THE INVENTION

In patients suffering sleep disruptions, one can observe a clear distinction between the ability to fall asleep and the ability to remain asleep long enough to feel rested. Pharmacological therapeutics for insomnia typically focus on enabling a patient to fall asleep. Most patients taking sleeping pills wake up in the middle of night without completing the 8-hour sleep cycle.

Previously the applicant filed a U.S. patent application (Ser. No. 14/614,916) disclosing a controlled-release, three-in-one composition (referred to as “SM-1” herein) for aiding sleep. The active ingredients of SM-1 include diphenhydramine HCl, zolpidem tartrate, and lorazepam. As proved in the Phase II clinical trial, SM-1 increases total sleep time in patients with transient insomnia. However, even though SM-1 contains the lowest effective dose of zolpidem tartrate and lorazepam, the side effects of these two drugs are still a concern.

The commonly reported side effects of zolpidem include dizziness, drowsiness, weakness, unsteady gait, difficulty with balance, uncontrollable shaking and headache, and it is associated with drug tolerance and substance dependence. In May 2013, the FDA approved label changes specifying lower dosage recommendations for zolpidem products because of concerns regarding next-morning impairment. Since zolpidem is metabolized by liver through CYP3A4, patients with liver problems should not take zolpidem as it can damage liver cells, thereby contributing to liver problems.

For lorazepam, common side effects may include dizziness, drowsiness, weakness, slurred speech, lack of balance or coordination, memory problems or feeling unsteady. Higher doses and longer periods of use increase the risk of developing a benzodiazepine dependence.

Therefore, there is a need for the improvement and optimization of SM-1 composition that allow a patient to fall asleep and stay asleep for a sufficient period of time with fewer side effects.

### SUMMARY OF THE INVENTION

Described herein is a controlled-release formulation for aiding sleep. The formulation comprises diphenhydramine HCl and a sleep aid which is zolpidem tartrate or lorazepam, wherein the formulation is formulated for releasing each drug at a specific time and a specific dose in a subject after the formulation is administered to the subject.

In one aspect, the formulation is formulated for two stages of release, wherein each stage initiates release of a drug in a subject at a specific time point after administration of the formulation to the subject. For example, the time interval between the initiations of each stage of release can be 0.5 to 23 hours. The first stage can be for immediate release of diphenhydramine HCl, and the second stage can be for delayed release of zolpidem tartrate or lorazepam.

In one aspect, the formulation is a tablet or capsule for oral administration. The tablet or capsule can contain a plurality of particles, each particle including a drug core and a polymeric composition encapsulating the core, wherein the drug core contains one or more drugs for aiding sleep.

According to certain embodiments of the present invention, the formulation comprises diphenhydramine HCl and a sleep aid which is zolpidem tartrate or lorazepam, as the sole active ingredients.

According to certain embodiments of the present invention, the formulation consisting essentially of diphenhydramine HCl and a sleep aid which is zolpidem tartrate or lorazepam.

Also described is a method of treating disturbed sleep or insomnia in a subject. The method includes administering the above-described formulation to a subject in need thereof.

It is to be understood that both the foregoing general description and the following detailed description are exemplary and explanatory only and are not restrictive of the invention.

### DETAILED DESCRIPTION OF THE INVENTION

### I. Introduction

It was unexpectedly discovered that administering the two-drug compositions (i.e., diphenhydramine HCl and zolpidem tartrate; or diphenhydramine HCl and lorazepam) as disclosed in the present application showed equivalent efficacy in maintaining sleep as compared to SM-1.

Through the elimination of one of the two active ingredients (zolpidem tartrate or lorazepam) from SM-1, the two-drug compositions can reduce the burden of drug intake in patients with transient insomnia, and therefore avoid the side effects as described above and metabolic burden of the eliminated drug.

Also, as one of the two active ingredients is eliminated from SM-1, the manufacturing cost for the two-drug compositions would be reduced.

### II. Definitions

The following abbreviations are used herein:

### AE: Adverse Event

### DSST: Digit Symbol Substitution Test

### KSS: Karolinska Sleepiness Scale

### PSG: Polysomnographic

### PSQ: Postsleep Questionnaire

sTST: subjective Total Sleep Time

### TST: Total Sleep Time

Unless defined otherwise, technical and scientific terms used herein have the same meaning as commonly understood by a person of ordinary skill in the art. Any methods, devices and materials similar or equivalent to those described herein can be used in the practice of this invention. The following definitions are provided to facilitate understanding of certain terms used frequently herein and are not meant to limit the scope of the present disclosure.

As used herein, the article “a” or “an” means one or more than one (that is, at least one) of the grammatical object of the article, unless otherwise made clear in the specific use of the article in only a singular sense.

As used herein, the term “disturbed sleep”, “sleep disturbances,” or “sleep disruption” refers to a condition characterized by waking up feeling unrestored, waking up in the middle of the night, difficulty returning to sleep after waking, difficulty falling asleep, and/or waking too early. Stress, a health condition, pain, a medication, jet lag, and noise are some factors that can lead to disturbed sleep. Disturbed sleep can be acute (i.e., short-termed) or chronic in duration.

An individual with insomnia experiences frequent and long-term disturbed sleep with daytime impairment or distress despite having adequate opportunity and circumstance for sleep.

Disturbed sleep can have various negative consequences such as fatigue, lack of energy, initiative reduction, daytime sleepiness, tension headache, gastrointestinal symptoms, irritability, anxiety, mood disturbance, reduced motivation, and impairment in cognitive functions (attention, concentration, and memory).

Whether a subject has disturbed sleep or insomnia can be determined by a skilled practitioner in the art.

As used herein, the term “treat” or “treating” refers to both therapeutic treatment and prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

The term “animal”, “subject” or “patient” as used herein includes, but is not limited to, humans and non-human vertebrates such as wild, domestic and farm animals, preferably humans.

### III. Embodiments of the Invention

Described herein is a controlled-release formulation comprising diphenhydramine HCl and a sleep aid which is zolpidem tartrate or lorazepam.

In one embodiment, the controlled-release formulation is formulated to release diphenhydramine HCl and a sleep aid selected from zolpidem tartrate and lorazepam. The formulation releases diphenhydramine HCl and the sleep aid at two stages. Each stage initiates release of diphenhydramine HCl and the sleep aid at a specific time after administration of the formulation.

For example, the formulation can release a first dose of diphenhydramine HCl immediately, release a second dose of the sleep aid 0.5-6 hour after release of the first dose.

The time interval between the initiation of each stage of release can be 30 minutes to 23 hours (e.g., 30 minutes, 1 hour, 1.5 hours, 2 hours, 3 hours, 3.5 hours, 4 hours, 4.5 hours, 5 hours, 5.5 hours, 6 hours, 6.5 hours, 7 hours, 8 hours, 9 hours, 10 hours, 12.5 hours, 13 hours, 15 hours, 20 hours, 22 hours, and 23 hours). Each stage can release a dose of a drug in the range of 0.01 mg to 100 mg (e.g., 0.01 mg, 0.05 mg, 0.1 mg, 0.25 mg, 0.4 mg, 0.5 mg, 0.6 mg, 1 mg, 2 mg, 3 mg, 4 mg, 5 mg, 6 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, and 100 mg).

The first stage of release can be an immediate release, in which release of diphenhydramine HCl is initiated shortly (e.g., within 30 minutes) after administration of the formulation. The first stage of release can also be a delayed released.

The above-described controlled-release formulation can be a tablet (e.g., a pill) or a capsule (e.g., a hard-shelled capsule or a softgel) for oral administration. Other formulations such as implants and patches can also be used.

Methods for formulating and making controlled-release formulations are known in the art. See, e.g., Hong Wen and Kinam Park, 2010, Oral Controlled Release Formulation Design and Drug Delivery: Theory to Practice, John Wiley & Sons, Inc. For example, controlled-release formulations can be designed based on particular physical mechanisms, e.g., dissolution, diffusion, osmosis, and ion exchange.

In a dissolution system, a drug (e.g., diphenhydramine HCl, zolpidem tartrate or lorazepam) is surrounded by or distributed in a polymeric composition (e.g., a polymeric membrane or a polymeric matrix). The drug is released when the polymeric composition dissolves. Properties of the polymeric composition, e.g., thickness and dissolution rate, determine drug release. In a diffusion system, the active ingredient has to diffuse through a polymeric composition (e.g., a polymeric membrane or a polymeric matrix) in order to be released. In an osmosis-based formulation, the drug is encapsulated by a polymeric coating that swells and erupts from osmotic pressure, thereby releasing the drug. Ion exchange formulation relies on attaching drug molecules to ionic groups. The drug molecules are then displaced by other ions and released. The controlled-release formulation described herein can utilize one release mechanism or a combination of release mechanisms.

In one embodiment, the above-described controlled-release formulation can be a tablet with multiple cores or layers. For example, the drug or combination of drugs for each stage of release can be surrounded by polymeric layer. The drug or combination of drugs is released as the layer dissolves.

In one embodiment, a multiparticulate system is employed. In this system, the drugs are each delivered in multiple particles (e.g., small beads or microspheres ranging from 0.05 to 3.00 mm in size), each particle exhibiting the desired characteristics (e.g., release time and rate). For example, the above-described controlled-release formulation can include a plurality of particles. Each particle contains a core including a drug or combination of drugs for aiding sleep and a controlled-release polymeric composition (containing one or more polymers) encapsulating the core. Properties of the controlled-release polymeric composition in each particle determine the drug release profile of each particle. The formulation can include uncoated particles for immediate release of a drug. Any of the above-described or other release mechanisms (e.g., dissolution, diffusion, and osmosis) can be employed in a multiparticulate system. The plurality of particles can be encapsulated in a capsule or compressed into a tablet for oral administration. For example, a three-stage release formulation can contain three types of particles, each type for each stage of release. Each dose of a drug for each stage of release is delivered by multiple particles.

Natural and synthetic polymers for controlled-release formulations are known in the art. Such polymers include, but are not limited to, proteins, polysaccharides, nucleotides, alginate, chitosan, heparin, xanthan gum, starch, pectin, gelatin, kit-carrageenan, hydroxypropylmethylcellulose, hydroxypropylcellulose, hydroxyethylcellulose, ethycellulose, methylcellulose, polyvinyl alcohol, polyacrylic acid, polyethylene oxide, poloxamers, pluronics, polymethacrylate, cellulose, collagen, nylon, polyalkylcyanoacrylate, polyethylene, polyethylene-co-vinylacetate, polyhydroxythyl methacrylate, polyhydroxypropylethyl methacrylate, polymethyl methacrylate, polyurethane, and silicon. Commercially available polymers for pharmaceutical applications include EUDRAGIT® polymethacrylates.

In one embodiment, the controlled-release formulation employs an enteric coating or other coatings for delaying drug release until the drug reaches the small intestine or the colon. Delaying drug release in this manner would also control drug release time. Such coatings are known in the art.

When a drug or combination of drugs is released, it becomes available to the body. Each stage of release can have a specific release rate. For example, a stage can have a pulsatile-release profile, in which a drug is released rapidly and completely following a period of no release. A stage can also have a first-order release rate, in which a drug is released at a decreasing release rate. A zero-order release rate, i.e., a constant release rate, can also be employed. An entire dose (or a significant portion thereof) of a drug can be released within a short period or over an extended period. For example, the formulation can be designed to release at least 50% (e.g., more than 60%, 70%, 75%, 80%, 85%, 90%, or 95%) of a dose of a drug or combination of drugs within 30 minutes of the initiation of release.

The controlled-release formulation can also include one or more pharmaceutical excipients, e.g., binders, plasticizers, lubricants, diluents, fillers, coloring agents, flavoring agents, glidants, and preservatives. Such pharmaceutical excipients are known in the art.

The controlled-release formulation can be administered to a patient daily or as needed to induce and maintains sleep.

### EXAMPLES

The present invention is more specifically explained by the following examples.

However, it should be noted that the present invention is not limited to these examples in any manner.

### Example 1—Three-Stage Release Capsule Formulation (SM-1)

Hard gelatin capsules for releasing diphenhydramine HCl (i.e., 2-(diphenylmethoxy)-N,N-dimethylethanamine hydrochloride), zolpidem tartrate (i.e., N,N,6-Trimethyl-2-p-tolylimidazo[1,2-a]pyridine-3-acetamide L-(+)-tartrate (2:1)), and lorazepam (i.e., (RS)-7-Chloro-5-(2-chlorophenyl)-3-hydroxy-1,3-dihydro-2H-1,4-benzodiazepin-2-one) were manufactured.

The capsules were designed to release the three drugs in a staged sequence controlled via pH-dependent coatings: uncoated diphenhydramine HCl for immediate release, coated zolpidem for release about 0.5-3 hours after administration, and coated lorazepam for release about 2-6 hours after administration.

SM-1 contained the following agents: (1) 50 mg of uncoated diphenhydramine HCl; (2) 5 mg of zolpidem tartrate in multiparticulate form coated with Eudragit® L100 D-55, which dissolves at above pH 5.5; and (3) 0.5 mg of lorazepam in multiparticulate form coated with Eudragit® L100/S100 (1:1), which dissolves at above pH 6.5. The coatings were selected to achieve release of zolpidem tartrate with gastric emptying and release of lorazepam when the drug reaches the intestines.

### Example 2—Two-Stage Release Capsule Formulation

The manufacture of two-stage release capsule formulations is the same as described above, except that lorazepam (or zolpidem tartrate) is eliminated from the formulation.

The diphenhydramine HCl plus zolpidem tartrate (D+Z) capsule contained the following agents: (1) 50 mg of uncoated diphenhydramine HCl; and (2) 5 mg of zolpidem tartrate in multiparticulate faun coated with Eudragit® L100 D-55, which dissolves at above pH 5.5.

The diphenhydramine HCl plus lorazepam (D+L) capsule contained the following agents: (1) 50 mg of uncoated diphenhydramine HCl; and (2) 0.5 mg of lorazepam in multiparticulate form coated with Eudragit® L100/S100 (1:1), which dissolves at above pH 6.5.

### Example 3—Efficacy Assessment for the Formulations in Human Subjects with Short-Term Insomnia

This study was a randomized, double-blind, single dose, 4-way crossover study to assess the efficacy of the capsules (as described in Examples 1 and 2 above) compared with placebo in a phase advance model of transient insomnia in subjects who reported previous transient insomnia. Sleep disturbance was induced using a 5-hour phase advance model with 8 hours (960 thirty-second epochs) of PSG recording as the primary efficacy assay. The study consisted of the following periods: 1. Screening period (Visit 1); 2. Treatment and PSG periods (Visits 2-5); and 3. Post-discharge follow-up safety phone call. Various parameters including total sleep time (TST) were assessed.

Screening Period (Visit 1)

Potential subjects were evaluated during a screening period lasting at least 8 and no more than 21 days before the first day of the first treatment period. After signed written informed consent was obtained, screening visit (Visit 1) procedures were performed. Subjects who satisfied all eligibility criteria were issued a pretreatment paper diary and instructed to record the time they went to bed with the intention of sleeping and the time they awakened for a minimum of 7 nights, with at least 5 entries completed over the 7 nights. Information recorded in the diary was communicated to study personnel no later than 24 hours before check-in for the first day of the first treatment period (Visit 2) to allow the calculation of each subject's median habitual bedtime. Before Visit 2, subjects were contacted by study personnel and told the time and date to present for their first overnight visit. Subjects were advised to eat lunch before check-in due to the long fasting intervals required by the study schedule.

A total of 157 subjects were screened and 85 subjected were randomly assigned to one of the 4 treatment sequences shown in Table 1.

TABLE 1
Treatment Sequences

| | Treatment | Treatment | Treatment | Treatment |
|---|---|---|---|---|
| | Period 1 | Period 2 | Period 3 | Period 4 |
| | Visit 2 | Visit 3 | Visit 4 | Visit 5 |
| Sequence 1 | SM-1 | D + Z | D + L | Placebo |
| Sequence 2 | D + Z | D + L | Placebo | SM-1 |
| Sequence 3 | D + L | Placebo | SM-1 | D + Z |
| Sequence 4 | Placebo | SM-1 | D + Z | D + L |

Abbreviations: D, diphenhydramine HCl; L, lorazepam; Z, zolpidem tartrate.
Note:
SM-1 = 50-mg diphenhydramine HCl, 5-mg delayed-release zolpidem tartrate, and 0.5-mg delayed-release lorazepam; D + Z = 50-mg diphenhydramine HCl and 5-mg delayed-release zolpidem tartrate; D + L = 50-mg diphenhydramine HCl and 0.5-mg delayed-release lorazepam; placebo = identical in appearance to SM-1, D + Z and D + L and had the same excipients, but no diphenhydramine HCl, zolpidem tartrate, lorazepam, or delayed-release coating materials.

Treatment and PSG Periods (Visits 2-5)

At Visit 2, subjects checked in at the study center approximately 7 hours earlier than their median habitual bedtime as calculated from their diary data and returned their paper diary to study personnel for review. Visit 2 procedures were performed and according to the timing of sleep study events relative to each subject's habitual bedtime. Each treatment and PSG period visit lasted approximately 11 to 12 hours. After the eligibility had been reconfirmed, subjects were randomly assigned to 1 of 4 treatment sequences as shown in Table 1.

Subjects went to bed (“Lights Out”) 5 hours (±30 minutes) before their median habitual bedtime. Approximately 90 minutes before Lights Out, baseline DSST measurements were obtained and subjects were provided a light, low-fat snack (e.g., fruit and crackers). Approximately 60 minutes before Lights Out, PSG electrodes were applied to the subject and the machine was calibrated. Subjects were administered their assigned treatment by study personnel 30 minutes before Lights Out, and an oral cavity check was performed to assure compliance with treatment. At the assigned Lights Out time the subject went to bed, PSG biocalibration was performed, and PSG recording began. The recording continued for 8 hours.

At the completion of the 8-hour PSG recording period, subjects were awakened and allowed a bathroom visit. Approximately 30 minutes after the end of the PSG recording, subjects completed the PSQ, KSS, and DSST and were served a standard breakfast. Before leaving the study center, subjects underwent a brief pre-discharge evaluation consisting of the tandem gait test, the Romberg test, and an assessment of vital signs and AEs. Subjects remained at the study center until they were able to pass the tests and could be discharged safely. Upon discharge, subjects were told the time and date to return for the next overnight visit, dispensed a daily washout interval diary, and instructed to maintain their normal sleep patterns.

Following a washout interval of no less than 5 days, subjects returned to the study center at the assigned time and date and returned their paper diary to study personnel for review. The schedule of procedures for Visits 3, 4, and 5 were the same as for Visit 2 except that subjects were given the next treatment in their randomization sequence (Table 1) and Lights Out occurred at the time established at Visit 2 (±15 minutes). Subjects were dispensed a new daily washout interval diary at Visits 3 and 4.

Follow-Up Safety Phone Call

Within 7 days after completion of Treatment Period 4 (Visit 5), but at least 72 hours after administration of the final dose, study personnel contacted the subject for a follow-up safety phone call. Outcomes of any AEs were discussed and recorded. If indicated, a visit to the study center for safety assessments was scheduled. From screening to the follow-up safety phone call, assuming a 7-day washout interval between each treatment period, subjects participated in the study for approximately 8 weeks.

Efficacy Results

Transient insomnia was successfully induced using the 5-hour phase advance model in this study, as evidenced by the decreased sTST in the placebo group (range: 45-540 minutes) compared to the sleep duration recorded in the sleep diary during the screening period (range: 330-670 minutes).

As shown in Table 2, treatments with SM-1, D+Z and D+L resulted in 95.8 minutes, 95.1 minutes and 90.3 minutes increased TST relative to placebo (P<0.0001), respectively. Differences between SM-1 and D+Z or D+L were not statistically significant.

TABLE 2
Summary of Total Sleep Time (TST) in Four Treatment Groups

| | | TREATMENT GROUP | | | | | | | |
|---|---|---|---|---|---|---|---|---|---|
| | | SM-1 | | D + Z | | D + L | | Placebo | |
| PARAMETER | CLASS | (N = 85) | | (N = 85) | | (N = 85) | | (N = 85) | |
| TST (mins) | Mean (SD) | 401.82 | (68.074) | 404.83 | (47.252) | 397.28 | (56.982) | 307.38 | (107.365) |
| | Median | 420.50 | | 417.00 | | 408.50 | | 312.00 | |
| | Min, Max | 131.0, 478.0 | | 244.0, 470.5 | | 150.0, 470.5 | | 28.5, 466.0 | |
| | LS Means (SE) | 406.5 | (7.76) | 405.8 | (7.77) | 401.0 | (7.77) | 310.7 | (7.79) |

| | | | | |
|---|---|---|---|---|
| p-value vs. SM-1 | | 0.9413 | 0.5600 | <0.0001 |
| p-value vs. Placebo | <0.0001 | <0.0001 | <0.0001 | |

In conclusion, SM-1, D+Z and D+L were all effective therapies in a 5-hour phase advance model of transient insomnia as evidenced in an increased TST in SM-1, D+Z and D+L treatments compared with placebo. SM-1, D+Z and D+L also showed a robust improvement in sleep maintenance. In addition, the observed efficacy (i.e., TST) did not differ between SM-1 and D+Z or D+L, indicating the elimination of lorazepam (D+Z) or zolpidem tartrate (D+L) can still maintain the same efficacy of SM-1.

While the foregoing written description of the invention enables one of ordinary skill in the art to make and use what is considered presently to be the best mode thereof, those of ordinary skill in the art will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

What is claimed is:

1. A controlled-release formulation, comprising diphenhydramine HCl and a sleep aid which is zolpidem tartrate or lorazepam, wherein the formulation is formulated for a first stage release of the diphenhydramine HCl immediately after administering to a subject, and a second stage release of the sleep aid that starts 0.5-6 hours after initiation of the first stage.

2. The formulation of claim 1, comprising 40-60 mg of diphenhydramine HCl.

3. The formulation of claim 1, comprising 4-6 mg of zolpidem tartrate.

4. The formulation of claim 1, comprising 0.4-0.6 mg of lorazepam.

5. The formulation of claim 2, comprising 50 mg of diphenhydramine HCl.

6. The formulation of claim 3, comprising 5 mg of zolpidem tartrate.

7. The formulation of claim 4, comprising 0.5 mg of lorazepam.

8. The formulation of claim 1, wherein the formulation is a tablet or capsule for oral administration.

9. The formulation of claim 8, wherein the tablet or capsule contains a plurality of particles, each particle including a drug core and a polymeric composition encapsulating the core, wherein the drug core contains zolpidem tartrate or lorazepam.

10. The formulation of claim 9, wherein the polymeric composition encapsulating the drug core containing zolpidem tartrate dissolves at above pH 5.5, or the polymeric composition encapsulating the drug core containing lorazepam dissolves at above pH 6.5.

11. The formulation of claim 9, wherein the polymeric composition includes a polymethacrylate.

12. The formulation of claim 1, comprising one or more pharmaceutical excipients.

13. A method of treating disturbed sleep or insomnia, comprising administering the formulation of claim 1 to a subject in need thereof.
